(19) United States
(12) Plant Patent Application Publication
(10) Pub. No.: US 2019/0037752 P1
(43) Pub. Date: Jan. 31, 2019

(54) Strawberry plant named 'Fresa Rica FNM'

(50) ;

(71) Applicant: Fresas Nuevos Materiales S.A., Huelva (ES)

(72) Inventor: Antonio Refoyo Piriz, Huelva (ES)

(21) Appl. No.: 15/998,037

(22) Filed: Jun. 19, 2018

(30) Foreign Application Priority Data
Jul. 26, 2017 (ES) 20175320

(51) Int. Cl. A01H 6/74

(52) U.S. Cl. PLT/208

(57) ABSTRACT

The present invention provides a new and distinct strawberry plant designated as ‘Fresa Rica FNM’, which originate from seed produced by manual cross between ‘E64’ and ‘86N’. The new strawberry is a day-neutral with a long, conical, glossy and a light red colour fruit. This new strawberry is characterized by a fruit of excellent quality, long shelf-life and high value of Brix degrees.

Latin name of genus and species: Fragaria x ananassa.

Varietal denomination: ‘Fresa Rica FNM’ (a.k.a., ‘A13-27N-3’). Inventor: Antonio Refoyo Píriz, Huelva (Spain). Assignee: Fresas Nuevos Materiales S.A.

### CROSS REFERENCE TO RELATED APPLICATIONS

This application claims priority to, and the benefit of the Spanish Plant Breeder's Right (PBR) Application No. 20175320, filed on Jul. 26, 2017, titled “Oficina Española de Variedades Vegetales”, directed to the strawberry variety in the present application (named as ‘A13-27N-3’ in the PBR application), which is herein incorporated by reference in its entirety.

Botanical designation: Fragaria x ananassa Duchesne.

Varietal denomination: ‘Fresa Rica FNM’.

### BACKGROUND OF THE INVENTION

The present invention relates to a new and distinct day-neutral strawberry variety designated as ‘A13-27N-3’ by the breeder, and later named ‘Fresa Rica FNM’.

‘Fresa Rica FNM’ was created in a breeding program and is the result of a controlled-cross between the experimental short day female parent designated ‘E64’ and day-neutral experimental selection ‘86N’, made by the Inventor and was first fruited in Gibraleón, (Huelva, Spain) growing fields. From the beginning and during testing, the plant was designated ‘A13-27N-3’.

This new variety was asexually reproduced via runners (stolons) by the inventor at Gibraleón, Huelva, Spain. Asexual propagules from the original source have been tested in Gibralcón, Moguer and Palos de la Frontera (Huelva, Spain) growing fields and to a limited extent, grower fields in high elevation in Arevalo, Avila, Spain. The properties of this variety were found to be transmissible by such asexual reproduction. This cultivar is stable and reproduces true to type in successive generations of asexual reproduction.

### SUMMARY OF THE INVENTION

The present invention relates to a new and distinct strawberry variety. The varietal denomination of the new variety is ‘Fresa Rica FNM’. Among the characteristics which are believed to distinguish the new variety from other varieties are a combination of traits which include the day length that is a day neutral; the plant growth habit it is upright and open with light density of foliage; the vigour of the plant is high, the inflorescence appears at level with the foliage; the fruit colour is medium red, the fruit is very sweet, conical-shaped, very firm, medium size fruit and long shelf-life.

### BRIEF DESCRIPTION OF THE DRAWING

The accompanying colour photographs and traits show typical specimens of the new cultivar at various stages of development as nearly true as it is possible to make in colour reproductions. The pictures were taken during in April in Huelva, Spain.

FIG. 1 shows overall plant habit including fruit at various stages of development.

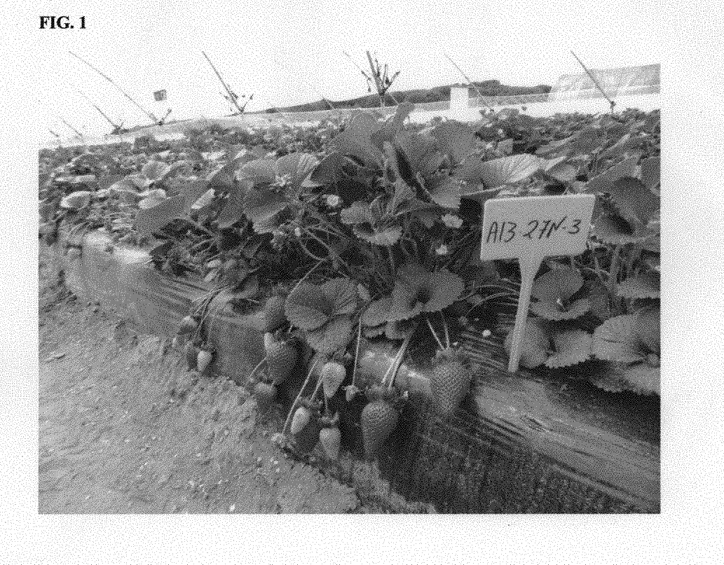

FIG. 2 shows leaves of the plant with three leaflets.

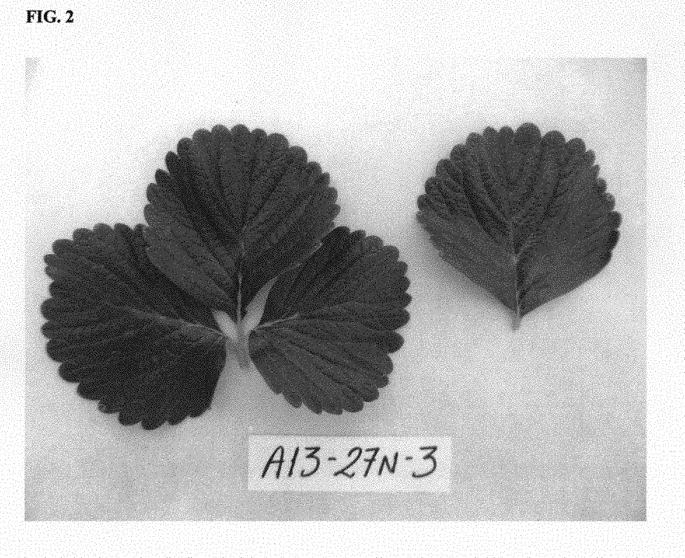

FIG. 3 shows the upperside of several flowers.

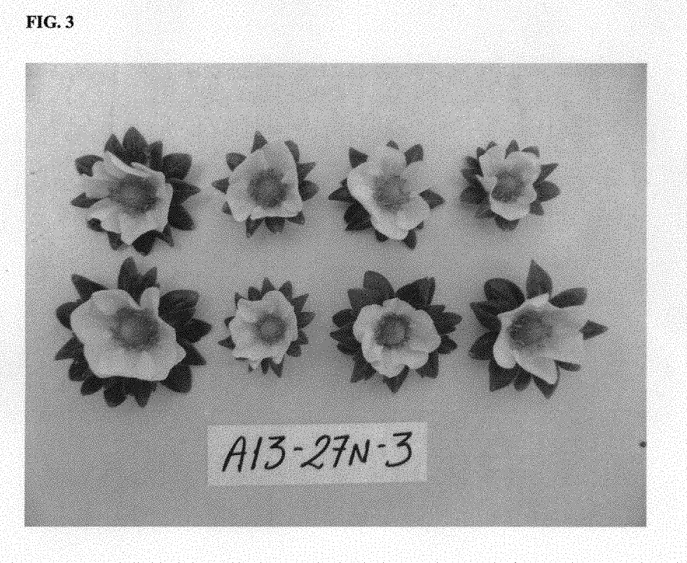

FIG. 4 shows the whole fruit.

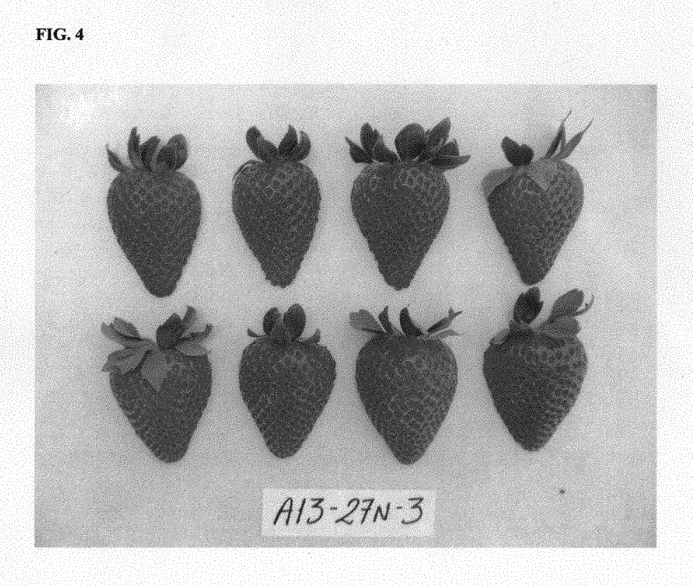

FIG. 5 shows the fruit in longitudinal cross-section.

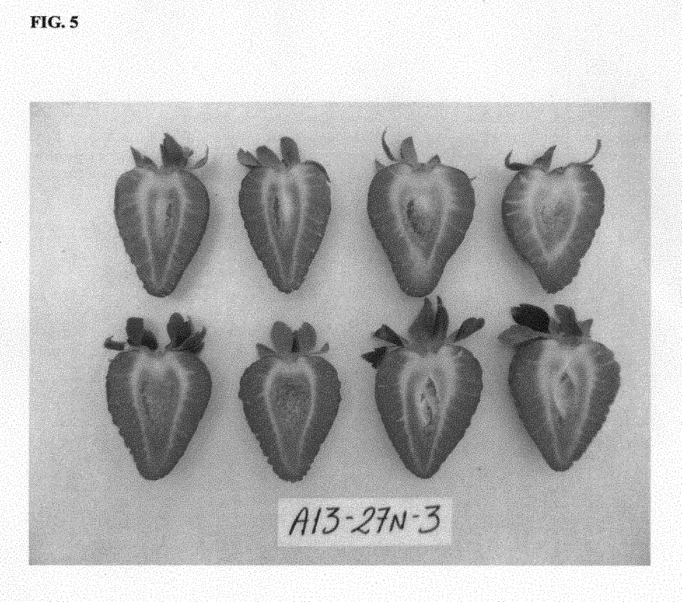

### DETAILED DESCRIPTION OF THE INVENTION

This invention relates to a new and distinctive day-neutral variety named ‘Fresa Rica FNM’. ‘Fresa Rica FNM’ produces fruit regardless of the day length when cultivated appropriately in temperate climates.

This cultivars is primarily adapted to the climate and growing conditions of the South of Spain (Huelva); Nevertheless considering that is a day-neutral cultivar, can planted in any place of the world that provides the necessary temperatures required for it to produce a strong vigorous plant and to remain in fruit production from March through October, this happen in the central coast of California where the nearby Pacific Ocean provides the needed humidity and moderate day temperatures and evening chilling to maintain fruit quality for the production months.

‘Fresa Rica FNM’ is a vigorous plant, upright and open which facilitates the fruit harvesting. It produces long and mainly unbranched pedunculus. ‘Fresa Rica FNM’ produces a fruit of a medium size, with a long conical shape, a medium red color and a glossy fruit. The fruit has an excellent quality, the firmness is high and its value of Brix degrees. The fruit of ‘Fresa Rica FNM’ has an excellent taste and appearance. Also noteworthy in the new variety ‘Fresa Rica FNM’ the great shelf-life that fruit presents. ‘Fresa Rica FNM’ produces a moderate amount of stolons which facilitates its multiplication.

‘Fresa Rica FNM’ is moderately resistant to the most frequent diseases in Huelva (Spain). Specifically, it is moderately resistant to powdery mildew (Sphaerotheca macularis), Verticillium wilt (Verticillium dahliae) and Phytophthora crown rot (Phytophthora cactorum). ‘Fresa Rica FNM’ is relatively tolerant to pests such as aphids and spider mites.

The results of production and Brix degrees of the variety ‘Fresa Rica FNM’ compared to the variety ‘San Andreas’ (U.S. Plant Pat. No. 19,975) are shown below (Table 1). The results that are shown come from a trial located in Gibraleón (Huelva, Spain). Trial was a randomized complete block design with three repetitions and 40 plants per plot. The planting date was Oct. 20, 2016 and was cultivated until May 25, 2017. It was cultivated under standard conditions of strawberry cultivation in Huelva, Spain.

TABLE 1

| Variety | pct | ppt | pcp | Brix | oid |
|---|---|---|---|---|---|
| Fresa Rica FNM | 1002 | 86 | 420 | 9, 7 | 3, 3 |
| San Andreas | 772 | 84 | 341 | 8, 8 | 3, 1 |
| LSD(5%) | 196 | 4 | 103 | 1, 0 | 0, 2 |
| LSD(1%) | 265 | 5 | 140 | 1, 3 | 0, 2 |

Fruit production and Brix degrees data of the new variety ‘Fresa Rica FNM’ and ‘San Andreas’.
Each value is the result of the average of three repetitions of 40 plants.
pct, total commercial yield in grams by plant;
ppt, percentage of firth class fruit;
pcp, early production (before March 31) in grams by plant;
brix, Brix degrees;
oid, powdery mildew (Sphaerotheca macularis) resistance score, 5 the best.
LSD(5%) and LSD(1%) Least Significant Difference between the means at P ≤ 0.05 and 0.01 respectively.

The fruit of ‘Albion’ has clear differences with respect to the fruit of the new variety ‘Fresa Rica FNM’. The variety ‘Albion’ shows a fruit with a darker red, a larger fruit and the more sunken achenes than the fruit of the variety ‘Fresa Rica FNM’.

The following traits in combination distinguish strawberry variety ‘Fresa Rica FNM’ from known strawberry varieties (Tables “I. General”, “II. Leaf”, “III. Petiole, the bract, and the stipule”, “IV. Inflorescence”, “V. Runner (Stolon)” and “VI. Fruit”). We use as standard variety for comparison ‘Albion’ (U.S. Plant Pat. No. 16,228). The traits were measured in Huelva (Spain), in April 2017. When we made the measurements, the plant was 6 months old. Fresh bare root plant was used for planting in October 2016. Plants for the botanical measurements in the present application were grown as annuals. Any colour references are made to The Royal Horticultural Society Colour Chart, 1995 Edition, except where general terms of ordinary dictionary significance are used.
I. General

| Characteristic | Fresa Rica FNM | Albion |
|---|---|---|
| Plant Growth Habit) | Upright | Semi-upright |
| Day length | Neutral | Neutral |
| Density of foliage | Light | Medium |
| Plant vigor | High | Moderate |
| Plant Growth Habit) | Upright | Semi-upright |
| Day length | Neutral | Neutral |
| Density of foliage | Light | Medium |
| Plant Vigor | High | Moderate |

II. Leaf

| Characteristic | Fresa Rica FNM | Albion |
|---|---|---|
| Terminal leaflet | 1.07 | 1.20 |
| length/width ratio | | |
| Leaf blistering | Medium | Weak |
| Terminal Leaflet margin | Serrate to crenate | Crenate |

III. Petiole, the bract, and the stipule

| Characteristic | Fresa Rica FNM | Albion |
|---|---|---|
| Petiole length (cm) | 19 | 17 |
| Petiole diameter (cm) | 0.33 | 0.42 |
| Petiole pose of hairs | Outwards | Slighty Outwards |
| Petiole color (color code) | RHS: N144C | RHS: N144A |
| Stipule length (cm) | 2.3 | 2.7 |
| Stipule width (cm) | 0.6 | 1.0 |
| Stipule pubescence | Weak | Weak |
| Stipule anthocyanin | Absence | Absence |
| presence or absence | | |
| Stipule color (color code) | RHS: 149C | RHS: N144C |
| Pedicel code (color code) | RHS: 144C | RHS: 144A |
| Attitude of hairs on | Upwards | Slightly Upwards |
| petiole and pedicel | | |

IV. Inflorescence

| Characteristic | Fresa Rica FNM | Albion |
|---|---|---|
| Inflorescence | Even | Below |
| position relative | | |
| to foliage | | |
| Petal spacing (e.g., overlapping etc.) | Overlapping | Touching |
| Petal number per flower | 5 | 6 |
| Upper and lower Petal color (color code) | RHS: NN155D | RHS: NN155D |
| peduncle size | Long | Medium |
| Sepal shape (e.g., Elliptical, etc.) | Elliptical | Elliptical |
| Sepal margin (e.g., entire, etc.) | Entire | Entire |
| Sepal number per flower | 12 | 10 |
| Number of stamen | 24 | 28 |
| Anther color (color code) | RHS: 2A | RHS: 2A |
| Time of flowering | November 30th | November 30th |
| Shape and color of stigma | Yellow | Orange |
| Color and length of style | Yellow, 1.9 mm | Green 1.2 mm |
| Color of the ovary | Green | White |
| Shape, size and color of anther | Oval, 1.9 mm, | Oval, 2.1 mm, |
| | Yellow | Yellow |
| Color and amount (scarce, moderate or | Yellow | Yellow |
| abundant) of pollen | Abundant | Abundant |
| Length and color of filament | 3.3 mm, Green | 4.2 mm, Green |

V. Runner (Stolon)

| Characteristic | Fresa Rica FNM | Albion |
|---|---|---|
| Stolon anthocyanin (color | RHS: N144D | RHS: N144B |
| code) | | |
| Stolon pubescence | Sparse | Medium |
| Stolon cancer (color code) | RHS: 149B | RHS: N144C |

VI. Fruit

| Characteristic | Fresa Rica FNM | Albion |
|---|---|---|
| Fruiting truss length | 11 cm | 7 cm |
| Truss color | RHS: 144A | RHS: N144A |
| Fruit length (cm) | 4.2 | 4.2 |
| Fruit width (cm) | 3.6 | 3.6 |
| Fruit skin color (color | RHS: 33A | RHS: 34A |
| code) | | |
| Fruit flesh color excluding | RHS: 33C | RHS: N25A |
| core | | |
| Fruit length/width ratio | 1.2 | 1.2 |
| Fruit weight (g) | 28 | 28 |
| Relative fruit size | Large | Large |
| Predominant fruit shape | conical | conical |
| Position of achenes | even | below |
| Achene color (color code) | RHS: 9A | RHS: 11A |
| Diameter of calyx | 3.6 | 3.9 |
| Color of calyx (color | RHS: 143A | RHS: 141A |
| code) | | |
| Color of the flesh | Light Red | Medium Red |
| Distribution of flesh color | Uniform | Some white center |
| Sweetness (by brix) | 9.3 | 7.4 |
| Type of bearing | day neutral | day neutral |
| Firmness | Firm | Medium |

The invention claimed is:

1. A new and distinct cultivar of strawberry plant having the characteristics substantially as described and illustrated herein.
